(19) United States
(12) Patent Application Publication
(10) Pub. No.: US 2022/0144902 A1
(43) Pub. Date: May 12, 2022

(54) METHOD FOR INDUCING MUSCULAR CELLS USING CELLS IN SPOT URINE

(71) Applicants: NATIONAL CENTER OF NEUROLOGY AND PSYCHIATRY, Tokyo (JP); NIPPON SHINYAKU CO., LTD., Kyoto (JP)

(72) Inventors: Hotake TAKIZAWA, Tokyo (JP); Yoshitsugu AOKI, Tokyo (JP); Shin'ichi TAKEDA, Tokyo (JP); Yukiko ENYA, Ibaraki (JP); Yuichiro TONE, Ibaraki (JP)

(21) Appl. No.: 17/418,203

(22) PCT Filed: Dec. 25, 2018
(86) PCT No.: PCT/JP2018/047447

§ 371 Date: Jun. 24, 2021

(51) Int. Cl. C07K 14/47; C12N 15/86; G01N 33/50; C12Q 1/6883; C12N 5/071

(52) U.S. Cl. CPC C07K 14/4702; C12N 15/86; C12Q 2600/158; C12Q 1/6883; C12N 5/0684; G01N 33/5044

(57) ABSTRACT

A method for preparing myotubes in a non-invasive and simple manner and establishes an in vitro test system of an agent used for exon skipping therapy for muscular dystrophy. The method can prepare myotubes from urine-derived cells by introducing the MYOD 1 gene into urine-derived cells and exposing the urine-derived cells to at least one epigenetic regulatory compound.

### TECHNICAL FIELD

The present invention relates to a method and a kit for preparing myotubes from urine-derived cells. Also, the present invention relates to a method for testing an agent used for exon skipping therapy of muscular dystrophy using the myotubes.

### BACKGROUND ART

Duchenne muscular dystrophy (DMD) is a serious hereditary muscular disease caused by dystrophin deficiency. For the treatment for DMD, practical application of exon skipping therapy using an antisense oligonucleotide (AON) has been expected. The exon skipping therapy is based on skipping an exon in the vicinity of a gene mutation by targeting an mRNA precursor with the use of AON (i.e., modification of abnormal splicing), modifying a frame-shift mutation to in-frame, and restoring the expression of a shortened dystrophin protein. As such agent used for exon skipping therapy, the inventors had developed the antisense oligonucleotide, NS-065/NCNP-01, that allows skipping of exon 53 of the dystrophin gene to restore dystrophin protein expression and completed the doctor-initiated early explorative test (Non-Patent Literature 1). At present, the next-phase testing is in progress. From now on, development of a novel exon skipping agent targeting an exon associated with a large number of target patients is expected.

It is also known that therapeutic effects cannot be always predicted at dystrophin mRNA and protein levels based on genomic DNA mutation patterns. Even when the exon skipping therapy is performed based on a particular genomic DNA mutation pattern, differences are sometimes observed in preferable dystrophin protein expression levels. In order to accelerate the development of an exon skipping agent for DMD treatment, select a subject on which therapeutic effects of such agent can be expected, and provide effective treatment to the subject, accordingly, it would be important to examine the effects of therapeutic agents using muscle cells derived from the subject in vitro prior to the initiation of the actual treatment.

### PRIOR ART LITERATURE

### Non-Patent Literature

Non-Patent Literature 1: Komaki, H. et al., Science translational medicine. vol. 10, eaan0713, 2018
Non-Patent Literature 2: Saito, T. et al, Plos One. vol. 5, e12239, 2010
Non-Patent Literature 3: Kim, E. Y. et al., Skeletal Muscle vol. 6: 32, 2016

### SUMMARY OF THE INVENTION

### Problems to be Solved by the Invention

In the past, the inventors had established the in vitro testing system comprising transforming the fibroblasts derived from the patient's skin into the myotubes via introduction of a muscle regulatory factor (i.e., MYOD1), and, after myotube differentiation, examining the effects of the exon skipping agent (Non-Patent Literature 2). However, a technique involving the use of dermal fibroblasts suffer from difficulties, such as the need for invasive skin biopsy and the need for performance of flow cytometry, which requires special equipment and techniques, in order to sort MYOD1-positive cells. In order to overcome such difficulties, development of an in vitro testing system of a therapeutic agent that can be performed in a non-invasive and simple manner is desired.

While a method of introducing MYOD1 into urine-derived cells to perform direct reprogramming into the myotubes has been reported (Non-Patent Literature 3), such technique had problems, such that cells with particular morphology were selected from among urine-derived cells in advance and it would take 4 to 5 weeks after the induction of differentiation in order to induce the myotubes.

### Means for Solving the Problems

The present inventors had focused on the urine-derived cells from the viewpoint of non-invasive testing and attempted the induction of the urine-derived cells into the myotubes by introducing the MYOD1 gene into the urine-derived cells as described in Non-Patent Literature 3. However, Myogenin, which is a muscle regulatory factor located downstream of MYOD1, was not substantially expressed, and myotubes could not be sufficiently induced. In order to overcome such problems, the present inventors had searched for the conditions in which the myotubes could be induced and succeeded in effective induction of the myotubes by exposing the urine-derived cells transduced with the MYOD1 gene to an epigenetic regulatory compound, such as a histone methyltransferase inhibitor (HMTI). We had also succeeded in testing the effects of an agent used for exon skipping therapy, which is a therapeutic agent for muscular dystrophy, with the use of the induced myotubes. The present invention had been completed based on such findings.

Specifically, the present invention encompasses the following aspects.
(1) A method for preparing myotubes from urine-derived cells comprising:
   a step of introducing the MYOD1 gene into urine-derived cells; and
   a step of exposing the urine-derived cells to at least one of epigenetic regulatory compounds.
(2) The method according to (1), wherein, after the introducing step and the exposing step, the urine-derived cells comprise at least one selected from the group consisting of myoblasts and myotubes.
(3) The method according to (1) or (2), wherein the epigenetic regulatory compound comprises at least one selected from the group consisting of a histone methyltransferase inhibitor, a histone demethylase inhibitor, a histone deacetylase inhibitor, a SIRT2 inhibitor, and a PARP inhibitor.
(4) The method according to (3), wherein the histone methyltransferase inhibitor comprises at least one selected from the group consisting of 3-deazaneplanocin A, 3-deazaneplanocin A hydrochloride (DZNep), GSK343, SGC707, furamidine dihydrochloride, UNC2327, E7438, and MI-2 (menin-MLL inhibitor).
(5) The method according to (3), wherein the histone demethylase inhibitor comprises at least one selected from the group consisting of IOX 1 and GSK-J1.
(6) The method according to (3), wherein the histone deacetylase inhibitor comprises at least one selected from the group consisting of LMK-235, CAY10603, BRD73954, and VORINOSTAT.
(7) The method according to (3), wherein the SIRT2 inhibitor comprises SirReal 2.
(8) The method according to (3), wherein the PARP inhibitor comprises EB47.
(9) The method according to any of (1) to (8), wherein the MYOD1 gene is introduced by introduction of an expression vector comprising the MYOD1 gene under the control of an inducible promoter.
(10) The method according to (9), wherein the expression vector further comprises a selection marker gene.
(11) The method according to any of (1) to (10), wherein the urine-derived cells are derived from a patient with a muscular disease or a patient with muscular dystrophy.
(12) A kit for preparing myotubes from urine-derived cells comprising:
   a means for introducing the MYOD1 gene into urine-derived cells; and
   at least one of epigenetic regulatory compound.
(13) The kit according to (12), wherein the introducing means is an expression vector used for introducing the MYOD1 gene into urine-derived cells.
(14) The kit according to (12) or (13), wherein the epigenetic regulatory compound comprises at least one selected from the group consisting of a histone methyltransferase inhibitor, a histone demethylase inhibitor, a histone deacetylase inhibitor, a SIRT2 inhibitor, and a PARP inhibitor.
(15) The kit according to any of (12) to (14), wherein the epigenetic regulatory compound comprises at least one selected from the group consisting of 3-deazaneplanocin A, 3-deazaneplanocin A hydrochloride (DZNep), GSK343, SGC707, furamidine dihydrochloride, UNC2327, E7438, MI-2 (menin-MLL inhibitor), IOX 1, GSK-J1, LMK-235, CAY10603, BRD73954, VORINOSTAT, SirReal 2, and EB47.
(16) A method for testing an agent used for exon skipping therapy for a patient with muscular dystrophy comprising:
   a step of preparing myotubes from urine-derived cells obtained from a patient with muscular dystrophy by the method according to any of (1) to (11);
   a step of applying the agent used for exon skipping therapy to the myotubes; and
   a step of detecting recovery of the dystrophin mRNA and/or protein in the myotubes.
(17) The method according to (16), wherein, in the detecting step, recovery of the dystrophin mRNA and/or protein is detected by at least one method selected from the group consisting of RT-PCR, Western blotting, and immunocytochemistry.
(18) The method according to (16) or (17), wherein the agent used for exon skipping therapy comprises at least one selected from the group consisting of an exon-44-skipping agent, an exon-45-skipping agent, an exon-50-skipping agent, an exon-51-skipping agent, and an exon-53-skipping agent.
(19) A method for screening for a candidate therapeutic agent or preventive agent of a condition of inducing skeletal muscle damage comprising:
   a step of preparing myotubes from urine-derived cells obtained from a patient with a condition of inducing skeletal muscle damage by the method according to any of (1) to (11);
   a step of applying a test substance or factor to the myotubes; and
   a step of identifying the test substance or factor as the candidate therapeutic agent or preventive agent by monitoring a change in the myotubes after the applying step.

### Effects of the Invention

The method and the kit according to the present invention enable induction of myotubes from urine-derived cells in a non-invasive and efficient manner With the use of the induced myotubes, the progress of fundamental studies involving the use of human-derived disease model muscle cells and the progress of personalized medicine provided for each patient developing myopathy, including muscular diseases and skeletal muscle damages, can be accelerated. Therefore, the present invention may be useful in the medical and drug discovery fields.

### BRIEF DESCRIPTION OF THE DRAWINGS

FIG. 1 shows an image of urine-derived cells that had formed colonies via urine culture obtained by phase contrast microscopy. The image was obtained 7 days after the initiation of primary culture.

FIG. 2 shows a retrovirus vector used to introduce the MYOD1 gene into urine-derived cells.

FIG. 3 shows a graph demonstrating a degree of muscular differentiation evaluated based on the expression level of the myosin heavy chain protein by immunocytochemistry. A horizontal axis represents a type of a compound added, and a vertical axis represents an area of a myosin heavy chain-positive region determined by immunocytochemistry (FIG. 3A: 1 μM low-molecular compound; FIG. 3B: 10 μM low-molecular compound).

FIG. 4 shows images demonstrating the effects of 3-deazaneplanocin A hydrochloride (DZNep) on promoting muscular differentiation analyzed by immunocytochemistry. Red represents a myosin heavy chain, and blue represents nuclear staining.

FIG. 5A shows blots and FIG. 5B shows graphs demonstrating the effects of 3-deazaneplanocin A hydrochloride (DZNep) on promoting muscular differentiation analyzed by Western blotting.

FIG. 6 shows the results of testing (RT-PCR) the effects of exon skipping therapy using the myotubes induced from urine-derived cells obtained from a DMD patient (the urine-cell derived myotubes). FIG. 6A shows dystrophin gene expression analyzed by RT-PCR, and FIG. 6B shows a graph demonstrating the exon skipping efficiency determined based on the results shown in FIG. 6A.

FIG. 7 shows the results of testing the effects of exon skipping therapy using the myotubes derived from urine-cell obtained from a DMD patient. FIG. 7A shows dystrophin gene expression analyzed by Western blotting, and FIG. 7B shows a graph prepared based on the results shown in FIG. 7A.

FIG. 8 shows the results of testing (immunocytochemistry) the effects of exon skipping therapy using the myotubes derived from urine-cell obtained from a DMD patient. Red represents a dystrophin protein, and blue represents nuclear staining.

FIG. 9 shows the results of the test system for selecting a sequence of an optimal agent used for exon skipping therapy. FIG. 9A shows dystrophin protein expression analyzed by immunocytochemistry, FIG. 9B shows a heat map for semi-quantitative analysis of fluorescence-positive regions based on FIG. 9A, and FIG. 9C shows a graph prepared based on FIG. 9B.

### EMBODIMENTS OF THE INVENTION

Hereafter, the present invention is described in detail.

The present invention relates to a method and a kit for preparing a urine-cell derived myotubes from urine-derived cells in a non-invasive and efficient manner and use of such urine-cell derived myotubes.

An aspect of the present invention relates to a method for preparing myotubes from urine-derived cells, and such method comprises: a step of introducing the MYOD1 gene into urine-derived cells; and a step of exposing the urine-derived cells to at least one of epigenetic regulatory compound. According to the method, induction of urine-derived cells into myotubes may be promoted by the introducing step and the exposing step.

In the present invention, a “myotube” means that expresses MYOD1 and is composed of a plurality of myoblasts fused to each other. Whether or not cells of interest are the myotubes can be evaluated in accordance with a method known in the art. For example, multinucleated cellular morphology may be observed, or the expression level of a muscle regulatory factor (e.g., MYOD1 or Myogenin), myosin, or dystrophin may be measured. Thus, whether or not cells of interest are the myotubes can be evaluated.

In the present invention, the term “urine-derived cells” is also referred to as “cells in spot urine” or “UDCs (urine-derived cells),” and the term refers to a cell population obtained by urine culture. While a urine sample before culture contains cells with various morphologies, such as renal epithelial cells or urothelial cells, as a result of cell proliferation through culture a relatively homogeneous cell population can be obtained (Zhou, T. et al., Generation of human induced pluripotent stem cells from urine samples, Nature protocols 7, 2080-2089, 2012).

The method of the present invention involves the use of urine-derived cells obtained by urine culture. While a urine source from which urine-derived cells are derived may vary depending on the purpose and the application after the myotube induction, a urine sample can be obtained from an animal, and preferably mammalian animals, such as a human, laboratory animal (e.g., mouse, rat, dog, or rabbit), or domestic animal (e.g., cattle or pig). According to a preferable embodiment, a urine source may be a human, and more preferably a human with a muscular disease caused by gene defect (e.g., muscular dystrophy).

Urine-derived cells can be obtained by a method known in the art (e.g., Zhou, T. et al., Nature protocols, vol. 7, pp. 2080-2089, 2012), and a method is not particularly limited. For example, a urine sample may be centrifuged to remove a supernatant, cell pellets may be mixed with the initial medium, incubated at approximately 37° C. and cultured in a growth medium, and cell colonies formed several days to about 2 weeks after the initiation of culture may then be selected. The cells thus obtained can be stable cell lines that can maintain similar properties after a plurality of times of passage culture.

According to the method of the present invention, the MYOD1 gene may be introduced into urine-derived cells. The MYOD1 gene is one of muscle regulatory factors and belongs to the MYOD family. When the MYOD1 gene is introduced into fibroblasts or the like, the cell can be induced to differentiate into myotubes. The MYOD1 gene and a method for introducing the gene into a cell have been well known in the art and are not particularly limited. Preferably, the MYOD1 gene of an animal from which urine-derived cells are derived, such as a human, may be used. The MYOD1 gene sequence, such as the human MYOD1 gene sequence, is registered to GenBank under Accession Number NM_002478.4.

The MYOD1 gene can be introduced into urine-derived cells by a method known in the art. Thus, the introducing step is performed. For example, the MYOD1 gene may be cloned and inserted into an appropriate expression vector (e.g., a retrovirus vector). In addition to the MYOD1 gene, a promoter, an enhancer, a selection marker gene, or the like may be inserted into an expression vector. A promoter can be appropriately selected in accordance with the origin of the urine-derived cells (e.g., a human origin), and use of an inducible promoter may be preferable. Upon MYOD1 expression, urine-derived cells initiate muscular differentiation, and the growth ability is decreased to a significant extent. Thus, it may be preferable that cell growth and differentiation into the myotubes be regulated with the use of an inducible promoter. Specifically, after introducing the MYOD1 gene into urine-derived cells using TRE3GS promoter as the inducible promoter, the urine-derived cells transduced with the MYOD1 gene may be allowed to grow, then the promoter may be activated with the addition of doxycycline (Dox) to the medium, and then the MYOD1 gene may be expressed such that the cells may be induced to differentiate into the myotubes. While a selection marker gene is not essential, it enables easy selection of the urine-derived cells transduced with the MYOD1 gene. Thus, a selection marker gene may preferably be inserted into an expression vector. Examples of selection marker genes include puromycin resistance gene, neomycin resistance gene, zeocin resistance gene, hygromycin resistance gene, and blasticidin resistance gene. Such expression vector may be introduced into urine-derived cells by a method known in the art with the use of a commercially available transfection reagent or the like. The cells containing the expression vector introduced thereinto can be selected in accordance with a method known in the art. When the puromycin resistance gene is inserted into an expression vector, for example, a cell having resistance to puromycin is to be selected.

According to the method of the present invention, urine-derived cells may be exposed to an epigenetic regulatory compound(s). Thus, the exposing step is performed. Specifically, urine-derived cells may be cultured in the presence of an epigenetic regulatory compound(s). According to an embodiment, the introducing step is followed by the exposing step. Alternatively, the introducing step may be performed simultaneously with or after the exposing step. Specifically, the MYOD1 gene may be introduced while or after urine-derived cells are cultured in the presence of an epigenetic regulatory compound(s) for a given period of time.

The term “epigenetic regulation” refers to regulation of gene expression via chromosome modification without modification of the nucleotide sequence of DNA. Examples of chromosome modification include chemical modification such as methylation of DNA in the nucleosome and acetylation and methylation of histone, and such chemical modification of DNA and histone regulates gene expression. Thus, the epigenetic regulatory compound(s) may include an inhibitor of an enzyme associated with such epigenetic regulation, such as an inhibitor of histone methyltransferase (HMT), histone demethylase, histone deacetylase (HDAC), SIRT2 (Sirtuin 2), or PARP (poly-ADP ribose polymerase).

The histone methyltransferase inhibitor is also referred to as “histone methyltransferase inhibitor” or “HMTI,” and it is a compound that inhibits histone methylation. Examples of appropriate histone methyltransferase inhibitors include 3-deazaneplanocin A, 3-deazaneplanocin A hydrochloride (DZNep), GSK343, SGC707, furamidine dihydrochloride, UNC2327, E7438, and MI-2 (menin-MLL inhibitor), with 3-deazaneplanocin A hydrochloride (DZNep), GSK343, furamidine dihydrochloride, UNC2327, and E7438 being preferable and 3-deazaneplanocin A hydrochloride (DZNep) being more preferable. Derivatives of such compounds having histone methyltransferase inhibitory activity can also be used.

A histone demethylase inhibitor is a compound that inhibits histone demethylation. Examples of appropriate histone demethylase inhibitors include IOX 1 and GSK-J1. Derivatives of such compounds having histone demethylase inhibitory activity can also be used.

A histone deacetylase inhibitor is also referred to as a histone deacetylase inhibitor or HDAC inhibitor, and it is a compound that inhibits histone deacetylation. Examples of appropriate histone deacetylase inhibitors include LMK-235, CAY10603, BRD73954, and VORINOSTAT, with LMK-235, CAY10603, and BRD73954 being preferable. Derivatives of such compounds having histone deacetylase inhibitory activity can also be used.

A SIRT2 (Sirtuin 2) inhibitor is a compound that inhibits SIRT2, and an example thereof includes SirReal 2. Derivatives of such compound having SIRT2 inhibitory activity can also be used.

A PARP (poly-ADP ribose polymerase) inhibitor is a compound that inhibits PARP, and an example thereof includes EB47. Derivatives of such compound having PARP inhibitory activity can also be used.

A single type of epigenetic regulatory compound may be used, or 2 or more types of compounds may be used in combination, for example, simultaneously or successively.

In the exposing step, the exposure conditions can be appropriately determined in accordance with a type of an epigenetic regulatory compound(s) used. Specifically, a medium, temperature, and the environment suitable for culture of urine-derived cells may be determined, the epigenetic regulatory compound(s) may be added to the medium, and urine-derived cells may be cultured therein. Examples of media that can be used may include, but are not limited to, a growth medium comprising the REGM Bullet Kit (Lonza; CC-3190) mixed with an equivalent amount of high-glucose DMEM, tetracyclin-free 15% fetal bovine serum, 0.5% Glutamax (Thermo Fisher Scientific; 35050-061), 0.5% non-essential amino acid (Thermo Fisher Scientific; 11140-050), 2.5 ng/ml fibroblast growth factor-basic (bFGF) (Sigma, St Louis, U.S.A.; F0291), PDGF-AB (Peprotech, Rocky Hill, N.J.; 100-OOAB), EGF (Peprotech; AF-100-15), 1% penicillin/streptomycin, and 0.5 μg/ml amphotericin B; and a differentiation medium comprising high-glucose-containing DMEM with GlutaMAX-I (Thermo Fisher Scientific; 10569-010), 5% horse serum, ITS Liquid Media Supplement (Sigma; 13146), and 1 μg/ml doxycycline. Such medium may be supplemented with an epigenetic regulatory compound(s) at appropriate concentration, such as a final concentration of 0.01 μM to 100 μM. A person skilled in the art can appropriately determine the condition of the epigenetic regulatory compound(s) in consideration of the myotube-inducing ability or cytotoxicity. Culture can be conducted at temperature suitable for mammalian animal cell culture, such as 30° C. to 40° C., and preferably approximately 37° C. and at around neutral pH. A culture period can be 1 hour to 4 weeks and preferably about 1 day to 2 weeks.

As a result of the introducing step and the exposing step, induction of urine-derived cells to differentiate into the myotubes may be promoted. Myotube induction can be confirmed by evaluating as to whether or not the cultured cell is the myotubes by, for example, measuring the expression level of the muscle regulatory factor (e.g., MYOD1 or Myogenin), myosin, or dystrophin and so on.

As described above, urine-derived cells may be sampled from the subject's urine, and myotubes can be prepared from the urine-derived cells. According to the method of the present invention, the myotubes can be prepared in a non-invasive and efficient manner. In this respect, accordingly, the method of the present invention is advantageous over conventional techniques.

The method described above can be performed in an easy and simple manner with the use of a kit. Specifically, another aspect of the present invention relates to a kit for preparing myotubes from urine-derived cells. This kit comprises a means for introducing the MYOD1 gene into urine-derived cells and at least one epigenetic regulatory compound. The introducing means may be, for example, the expression vector used for introducing the MYOD1 gene into urine-derived cells as described above. The epigenetic regulatory compound(s) may be provided together with a medium suitable for induction of differentiation into the myotubes. The kit may comprise, as components, the introducing means and an epigenetic regulatory compound(s), and the components may further comprise instructions describing the procedure and the protocol for implementing the method described above.

Components of the kit may be individually and separately provided, or components may be accommodated in a single container and provided in that state. Preferably, the kit comprises all components necessary to perform the method described above at adjusted concentration, so that the kit can be used immediately.

The myotubes prepared by the method or by the use of the kit described above can be used to evaluate the effects of a therapeutic agent for a condition of inducing skeletal muscle damage. For example, the myotubes prepared by the method or by the use of the kit described above can be used for evaluation of the effects of an agent used for exon skipping therapy for a patient with muscular dystrophy and/or screening of a candidate therapeutic agent or preventive agent for the condition of inducing skeletal muscle damage.

Specifically, another aspect of the present invention relates to a method for testing a therapeutic agent for the condition of inducing skeletal muscle damage. The method for testing comprises:

a step of preparing myotubes from urine-derived cells obtained from a patient with a condition of inducing skeletal muscle damage by the method described above;

a step of applying a therapeutic agent to the myotubes; and

a step of detecting an improvement in the condition of skeletal muscle damage in the myotubes. More specifically, the present invention relates to a method for testing an agent used for exon skipping therapy for a patient with muscular dystrophy comprising:

a step of preparing myotubes from urine-derived cells obtained from a patient with muscular dystrophy by the method described above;

a step of applying the agent used for exon skipping therapy to the myotubes; and

a step of detecting recovery of the dystrophin mRNA and/or protein in the myotubes after the applying step.

According to the method for testing, the term a “condition of inducing skeletal muscle damage” is a generic term indicating a condition in which various symptoms are developed upon myogenic or neurogenic damage of the muscle. Examples thereof may include congenital muscular dystrophies, such as Duchenne muscular dystrophy, Becker muscular dystrophy, Fukuyama congenital muscular dystrophy, merosin-deficient congenital muscular dystrophy, and Ullrich congenital muscular dystrophy; neuromuscular junction disorders, such as myopathy, inflammatory muscular disease, and myasthenic syndrome; neurodegenerative disorders, such as amyotrophic lateral sclerosis; peripheral nerve disorders, such as myelopathic muscular atrophy; diseases that induce disuse atrophy including after effects of cerebral stroke; sarcopenia; and cancer cachexia.

The method for testing according to the present invention comprises preparing myotubes from urine-derived cells obtained from a patient with a condition of inducing skeletal muscle damage. Thus, the preparing step is performed. A patient with a condition of inducing skeletal muscle damage may be a human patient actually having the skeletal muscle damage or a condition of inducing skeletal muscle damage, and such patient may preferably be a candidate human patient to which the test therapeutic agent is to be administered. According to the method described above, a urine sample may be obtained from a patient with a condition of inducing skeletal muscle damage, urine-derived cells may be obtained therefrom, and the myotubes derived from the patient may be prepared.

Subsequently, the test therapeutic agent may be applied to the myotubes prepared above. Thus, the applying step is performed. A therapeutic agent may not be particularly limited, provided that it is used for treatment of the skeletal muscle damage or a condition of inducing skeletal muscle damage. For example, the use of an agent used for exon skipping therapy, a read-through therapeutic agent, and gene therapy with a virus vector have been known as the therapy for muscular dystrophy. An agent used for exon skipping therapy is a therapeutic agent that recovers the expression of shortened dystrophin protein by skipping an exon in the vicinity of a genetic mutation by targeting a dystrophin mRNA precursor using an antisense oligonucleotide (AON) and modifying a frame-shift mutation into in-frame. For example, an exon-44-skipping agent, an exon-45-skipping agent, an exon-50-skipping agent, an exon-51-skipping agent, and an exon-53-skipping agent are known, and the AON sequences thereof are also known (see, for example, Wilton, S. D. et al., Mol. Ther., 15, 1288-1296, 2007 for the exon-44-skipping agent, the exon-45-skipping agent, and the exon-53-skipping agent; Wu, B. et al., PLoS One 6, e19906, 2011 for the exon-50-skipping agent, and eteplirsen (AVI-4658) for the exon-51-skipping agent). In the method for testing, a single therapeutic agent may be tested, or a plurality of therapeutic agents may be simultaneously tested to compare the effects of the therapeutic agents.

A person skilled in the art can readily determine the conditions in which the therapeutic agent is applied. For example, the myotubes may be cultured in a medium supplemented with the therapeutic agent for a given period of time, such as for 1 hour to 5 days. The effects and the efficacy of the therapeutic agent can be tested under several conditions. Examples of conditions include the time at which the therapeutic agent is applied, the amount of the therapeutic agent to be applied, and the number of times the therapeutic agent is applied.

Subsequently, an improvement in the condition of skeletal muscle damage in the myotubes may be detected. Thus, the detecting step is performed. The condition to be detected varies depending on a type of skeletal muscle damage or a condition of inducing skeletal muscle damage. In the case of muscular dystrophy having a deficiency in the dystrophin protein expression in muscle cells, for example, recovery of the dystrophin mRNA and/or protein in the myotubes may be detected. Recovery of dystrophin can be detected by a method known in the art. Specifically, recovery can be detected at the mRNA level (e.g., by RT-PCR) or at the protein level (e.g., by Western blotting or immunocytochemistry). For comparison, the effects of the therapeutic agents may be compared with the use of, for example, the myotubes to which no therapeutic agent has been applied or the myotubes derived from a healthy subject (such myotubes may preferably be induced from urine-derived cells by the same technique).

According to the method for testing, the effects of the therapeutic agents on a condition of inducing skeletal muscle damage, and, in particular, on muscular dystrophy, can be evaluated. More specifically, a therapeutic agent for a patient with particular skeletal muscle damage or a condition of inducing skeletal muscle damage (a patient with muscular dystrophy) can be tested, and a therapeutic agent that is predicted to be highly effective can be selected.

Another aspect of the present invention relates to a method for screening for a candidate therapeutic agent or preventive agent for a condition of inducing skeletal muscle damage.

The method for screening according to the present invention comprises:
a step of preparing myotubes from urine-derived cells obtained from a patient with a condition of inducing skeletal muscle damage by the method described above;
a step of applying a test substance or factor to the myotubes; and
a step of identifying the test substance or factor as the candidate therapeutic agent or preventive agent by monitoring a change in the myotubes after the applying step.

According to the method for screening, the term a “condition of inducing skeletal muscle damage” is a generic term indicating a condition in which various symptoms are developed upon myogenic or neurogenic damage of the muscle. Examples thereof may include congenital muscular dystrophies, such as Duchenne muscular dystrophy, Becker muscular dystrophy, Fukuyama congenital muscular dystrophy, merosin-deficient congenital muscular dystrophy, and Ullrich congenital muscular dystrophy; neuromuscular junction disorders, such as myopathy, inflammatory muscular disease, and myasthenic syndrome; neurodegenerative disorders, such as amyotrophic lateral sclerosis; peripheral nerve disorders, such as myelopathic muscular atrophy; diseases that induce disuse atrophy including after effects of cerebral stroke; sarcopenia; and cancer cachexia.

The method for screening comprises preparing myotubes from urine-derived cells obtained from a patient with a condition of inducing skeletal muscle damage. A patient with a condition of inducing skeletal muscle damage may be a human patient actually having the condition of inducing skeletal muscle damage or an animal model of the condition of inducing skeletal muscle damage. For example, mouse models of muscular dystrophy (mdx mice), dog models (GRMD and CXMDJ dogs), and cat models (HFMD cats) are known. A urine sample may be obtained from a patient with a condition of inducing skeletal muscle damage or an animal model thereof, urine-derived cells may be obtained therefrom, and the myotubes derived from the patient or animal model may be prepared. Thus, the preparing step is performed.

According to the method for screening, the target test substances or factors are not particularly limited. For example, test substances or factors may be any substances. Specific examples include: naturally-occurring molecules, such as amino acids, peptides, oligopeptides, polypeptides, proteins, nucleic acids, lipids, carbohydrates (e.g., sugar), steroids, glycopeptides, glycoproteins, and proteoglycans; synthetic analogs or derivatives of naturally-occurring molecules, such as peptide mimics and nucleic acid molecules (e.g., aptamers, antisense nucleic acids, an agent used for exon skipping therapy, and double-stranded RNA (RNAi)); non-naturally occurring molecules, such as low molecular organic compounds prepared using a combinatorial chemistry technique (e.g., inorganic and organic compound libraries or combinatorial libraries); and mixtures of any thereof. The test substance or factor may be a single substance, it may be a complex or composite of a plurality of substances, or it may be transcription factors or the like. In addition, factors may be environmental factors, such as radiation, ultraviolet, oxygen or carbon dioxide concentration, or temperature.

In the method for screening, the test substance or factor may be applied to the myotubes. A person skilled in the art can readily determine the conditions. For example, the myotubes may be cultured in a medium supplemented with the test substance, the myotubes may be soaked in a solution containing the test substance, the test substance may be overlaid on the myotubes, or the myotubes may be cultured in the presence of the test factor. Thus, the applying step is performed.

The effects and the efficacy of the test substance or factor can be tested under several conditions. Examples of conditions include the time at which the test substance or factor is applied, the duration during which the test substance or factor is applied, the amount of the test substance or factor to be applied, and the number of times the test substance or factor is applied. For example, a dilution series of the test substance may be prepared to determine a plurality of doses. The duration for treatment with the test substance or factor can be appropriately determined. For example, such treatment can be performed over the period of 1 hour to several days, several weeks, several months, or several years.

When additive action, synergistic action, and other action of a plurality of test substances and/or factors are to be examined, in addition, test substances and/or factors may be used in combination.

Subsequently, a change in the myotubes may be monitored. A change to be monitored varies depending on conditions of inducing skeletal muscle damage. In the case of muscular dystrophy having a deficiency in the dystrophin protein expression in muscle cells, for example, expression of the dystrophin protein in the myotubes may be monitored. After a change in the myotubes is monitored, the results of monitoring may be compared with the results of the control samples, and the test substance or factor that can improve the condition of the skeletal muscle damage may then be selected as a candidate therapeutic agent or preventive agent. For comparison, the myotubes in the absence of the test substance or factor or the myotubes derived from a healthy subject (such myotubes may preferably be induced from urine-derived cells by the same technique) can be used. Thus, the identifying step is performed.

Upon screening for a candidate therapeutic agent or preventive agent, in addition, the selected test substance or factor may be administered to an animal model of skeletal muscle damage or a condition of inducing skeletal muscle damage (i.e., an animal that developed skeletal muscle damage or an animal that carries skeletal muscle damage) to evaluate as to whether or not the test substance or factor would influence the pathological conditions of the skeletal muscle damage in the animal model. Whether or not the test substance or factor would influence the pathological conditions of the skeletal muscle damage in the animal model can be evaluated depending on, for example, a skeletal muscle damage type, an animal model type, a pathological condition to be evaluated, or a causal factor. A person skilled in the art can appropriately evaluate the influence on the skeletal muscle damage. In the case of muscular dystrophy, for example, measurement of the muscle strength, measurement of the serum creatine kinase level, measurement of the tension of the isolated skeletal muscle, histological measurement of the maximal muscle diameter, or measurement of the frequency of the central nuclear fiber can be performed. In general, the efficacy of the test substance or factor is first verified in the animal model, and the efficacy is then evaluated via, for example, clinical trial in a human.

As described above, the test substance or factor can be selected as a candidate therapeutic agent or preventive agent for skeletal muscle damage or a condition of inducing skeletal muscle damage when an improvement is observed in the condition of inducing skeletal muscle damage (e.g., an improvement in symptoms or delay in the development or advancement of symptoms). For example, the test substance or factor that improves muscular dystrophy symptoms (e.g., lowered muscle strength, muscle atrophy, lowered motor ability, gait disturbance, and myocardial disease) or that delays the development or advancement of symptoms is to be selected.

### EXAMPLES

Hereafter, the present invention is described in further detail with reference to the examples and the drawings. It should be noted that the present invention is not limited to the examples described below.

All the experiments described in the examples were performed upon receipt of approval from the National Center of Neurology and Psychiatry (NCNP). Spot urine samples were obtained upon receipt of consent in writing from donors or proxies.

### [Example 1] Sampling and Culture of Urine-Derived Cells

Urine samples were obtained by having the subjects to urinate in sterilized plastic bottles (Corning Incorporated, NY, U.S.A.; 430281). The method of Zhou et al. (Zhou, T. et al., Nature protocols, vol. 7, pp. 2080-2089, 2012) was appropriately modified, and urine samples were subjected to the primary cell culture within several hours after sampling.

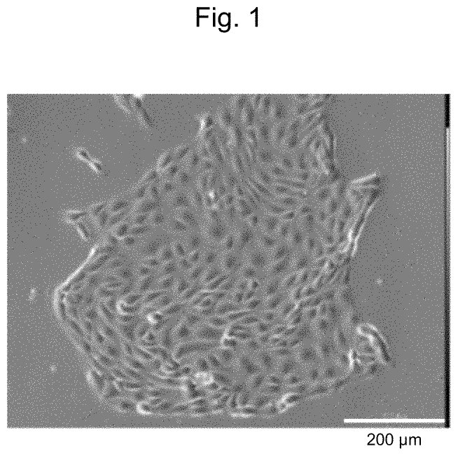

Briefly, the urine samples were aliquoted into a plurality of 50-ml conical tubes, the urine samples were centrifuged at 400×g at room temperature for 10 minutes, and the supernatant was then removed. Thereafter, the pellets were suspended in PBS and collected in a conical tube. A wash solution (10 ml, Ca²⁺- and Mg²⁺-free PBS containing 1% penicillin/streptomycin (Thermo Fisher Scientific, Waltham, Mass.; 15140-122) and 0.5 μg/ml amphotericin B (Sigma, St Louis, U.S.A.; A2942)) was added to the conical tube, the resultant was centrifuged at 200×g at room temperature for 10 minutes, and the supernatant was then removed. The pellets were suspended in 1.5 ml of the initial medium (high-glucose DMEM (GE Healthcare, Logan, Utah; SH30022.FS) was mixed with an equivalent amount of Ham's F-12 Nutrient Mix (Thermo Fisher Scientific; 11765-054), and REGM SingleQuots (Lonza, Basel, Switzerland; CC-4127), tetracyclin-free 10% fetal bovine serum (Clontech; 631106), 1% penicillin/streptomycin, and 0.5 μg/ml amphotericin B were added), and the cell suspension was cultured on a gelatin-coated 6-well plate (IWAKI, Shizuoka, Japan; 4810-020) in an incubator in the presence of 5% CO₂ at 37° C. The initial medium was added in an amount of 1.5 ml each every day, and the culture medium was substituted with 2 ml of the growth medium (the medium containing the REGM Bullet Kit (Lonza; CC-3190) mixed with an equivalent amount of high-glucose DMEM, tetracyclin-free 15% fetal bovine serum, 0.5% Glutamax (Thermo Fisher Scientific; 35050-061), 0.5% non-essential amino acid (Thermo Fisher Scientific; 11140-050), 2.5 ng/ml fibroblast growth factor-basic (bFGF) (Sigma, St Louis, U.S.A.; F0291), PDGF-AB (Peprotech, Rocky Hill, N.J.; 100-OOAB), EGF (Peprotech; AF-100-15), 1% penicillin/streptomycin, and 0.5 μg/ml amphotericin B, provided that amphotericin B/gentamicin of the REGM Bullet Kit is excluded) 4 days after the initiation of culture. The urine-derived cells formed colonies several days to about 2 weeks after the initiation of culture. FIG. 1 shows an image obtained by phase contrast microscopy 7 days after the initiation of culture.

### [Example 2] Preparation of Retrovirus Vector

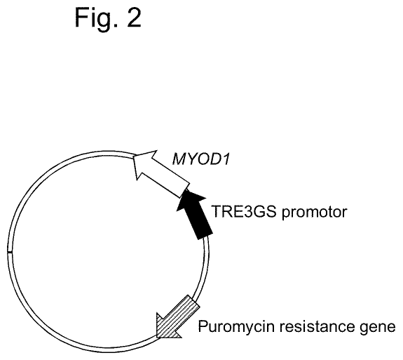

With the use of In-Fusion HD Cloning Plus (Clontech; 638909), the MYOD1 sequence (CCDS 7826.1) was inserted into the pRetroX-TetOne-Puro vector (Clontech; 634307). GP2-293 cells (Clontech; 631458) were cultured on a collagen-coated cell culture plate in a DMEM medium containing 10% fetal bovine serum. With the use of Xfect transfection reagent (Clontech; 631317), the pVZV-G capsid vector and the pRetroX-TetOne-Puro vector containing MYOD1 inserted therein were transfected into the GP2-293 cells. A retrovirus vector produced in the GP2-293 cells (hereafter referred to as the “MYOD1 virus vector,” FIG. 2) was recovered from the culture supernatant after 24 hours and 48 hours and stored in a freezer at −80° C. In the retrovirus vector shown in FIG. 2, the MYOD1 gene is under the control of the TRE3GS promoter. Thus, the expression of MYOD1 gene can be induced by doxycycline (Dox). In addition, the vector contains the puromycin resistance gene as a selection marker.

### [Example 3] Introduction of MYOD1 into Urine-Derived Cells

The urine-derived cells were plated onto a culture dish or plate (e.g., 3,000 to 5,000 cells/cm²), cultured in a growth medium, and infected with the MYOD1 virus vector using polybrene or the like (e.g., after 24 hours) to introduce MYOD1 into the urine-derived cells. Thus, the introducing step was performed. After a given period of infection, puromycin was added to the medium, culture was conducted for several days, and MYOD1-positive urine-derived cells were then selected.

### [Example 4] Promotion of Induction of Urine-Cell Derived Myotubes Via Exposure to Low Molecular Compound

The MYOD1-positive urine-derived cells were plated onto a collagen-coated culture dish or plate and cultured in a differentiation medium supplemented with doxycycline (e.g., 1 μg/ml) (the medium containing high-glucose-containing DMEM with GlutaMAX-I (Thermo Fisher Scientific; 10569-010), 5% horse serum, ITS Liquid Media Supplement (Sigma; 13146), and 1 μg/ml doxycycline) to induce the myotubes. Whether or not muscular differentiation could be promoted with the addition of a low molecular compound in a compound library (Sigma; S990043-EPI1) to the differentiation medium was examined. The low molecular compound was added at a final concentration of 0.1, 1, or 10 μM. Myotube induction was evaluated by immunocytochemistry and Western blotting.

For immunocytochemistry, cultured cells were washed in PBS, fixed in 4% paraformaldehyde, and then incubated with the addition of 0.1% Triton-X at room temperature for 10 minutes. The anti-myosin heavy chain antibody (1:50, R&D, Minneapolis, U.S.A.; MAB4470) and the anti-dystrophin antibody (1:30, Novocastra, Newcastle, UK; NCL-DYS1) were used as primary antibodies, and Alexa Fluor 546 goat anti-mouse IgG (H+L) (1:300, Invitrogen; A11003) was used as a secondary antibody. Nuclear staining was performed with the use of Hoechst 33342. An image was obtained using a fluorescence microscope (BZ-9000 or BZ-X800, KEYENCE, Osaka, Japan) and analyzed using the BZ-X Analyzer (KEYENCE).

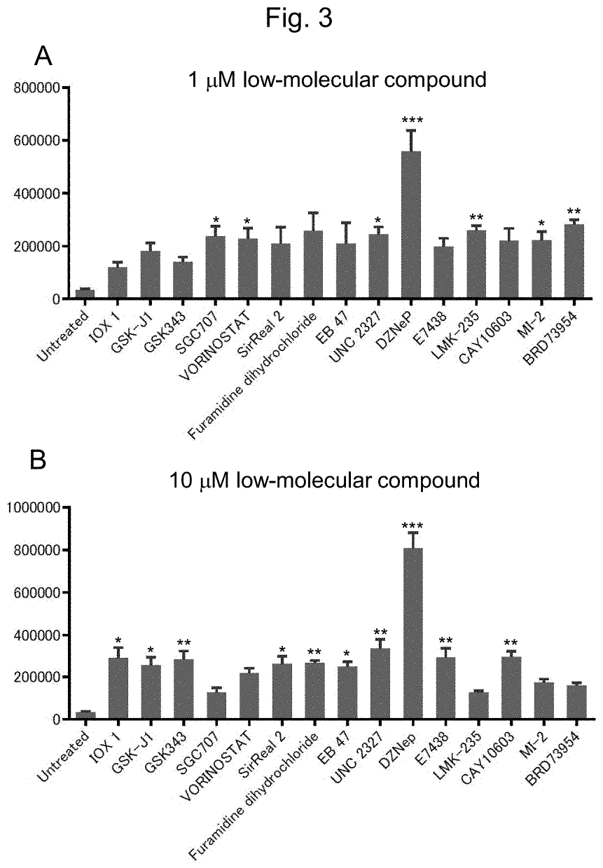

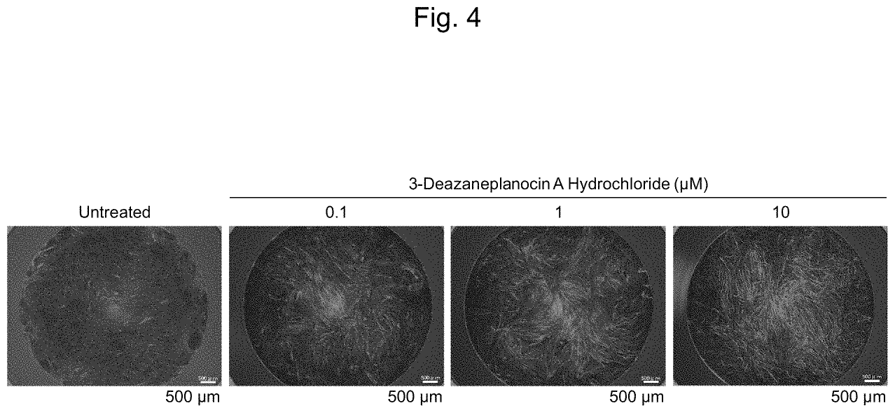

As a result, it was founded that a degree of muscular differentiation evaluated in terms of the myosin heavy chain protein expression level by immunocytochemistry was enhanced to a significant extent with the addition of the epigenetic regulatory compound(s) to the differentiation medium (FIGS. 3 and 4). In particular, effects of a histone methyltransferase inhibitor, 3-deazaneplanocin A hydrochloride (hereafter, referred to as “DZNep”), were found to be high. Also, effects of histone methyltransferase inhibitors (GSK343, SGC707, furamidine dihydrochloride, UNC2327, E7438, and MI-2 (menin-MLL inhibitor)), histone demethylase inhibitors (IOX 1 and GSK-J1), histone deacetylase (HDAC) inhibitors (VORINOSTAT, LMK-235, CAY10603, and BRD73954), SIRT2 inhibitor (SirReal 2), and PARP inhibitor (EB47) were observed. In FIG. 3, a horizontal axis represents a type of a compound added, and a vertical axis represents an area of a myosin heavy chain-positive region determined by immunocytochemistry. FIG. 3A shows the results obtained with the use of a 1 μM low-molecular compound, and FIG. 3B shows the results obtained with the use of a 10 μM low-molecular compound. Statistical analysis was performed by a Kruskal-Wallis test at a significance level of p<0.05. “*,” “**,” and “***” indicate p<0.05, p<0.01, and p<0.001, respectively.

The effects of DZNep on promoting myotube induction were also examined by Western blotting. Specifically, Western blotting was performed in the manner described below. The cells were lysed in a RIPA buffer (Thermo Fisher Scientific; 89900) containing a protease inhibitor (Roche, Indianapolis, Ind., U.S.A.; 04693116001), the cell lysate was centrifuged at 4° C. and 14,000×g for 15 minutes, and the supernatant was then recovered. The total protein concentration was measured using the BCA protein assay kit (Thermo Fisher Scientific; 23227), denaturation was performed using NuPAGE® LDS Sample Buffer (Thermo Fisher Scientific; NP0007), SDS-PAGE was performed on 3% to 8% NuPAGE® Novex Tris-Acetate Gel (Invitrogen; EA03785BOX), and the resultant was transferred onto a PVDF membrane (Millipore, Billerica, Mass., U.S.A.; IPVH304F0). The antibody reaction was conducted by using, as primary antibodies, rabbit anti-dystrophin antibody (1:500, Abcam, Cambridge, UK; ab15277), mouse anti-myosin heavy chain antibody (1:200, R&D, Minneapolis, U.S.A.; MAB4470), and mouse anti-α-tubulin antibody (1:1000, Sigma; T6199) and, as a secondary antibody, Histofine Simple Stain MAX-PO (1:100, NICHIREI BIOSCIENCE INC., Tokyo, Japan; 424151). After the antibody reaction, a band of interest was detected using the ECL Prime Western Blotting Detection Reagent (GE Healthcare, UK; RPN2232).

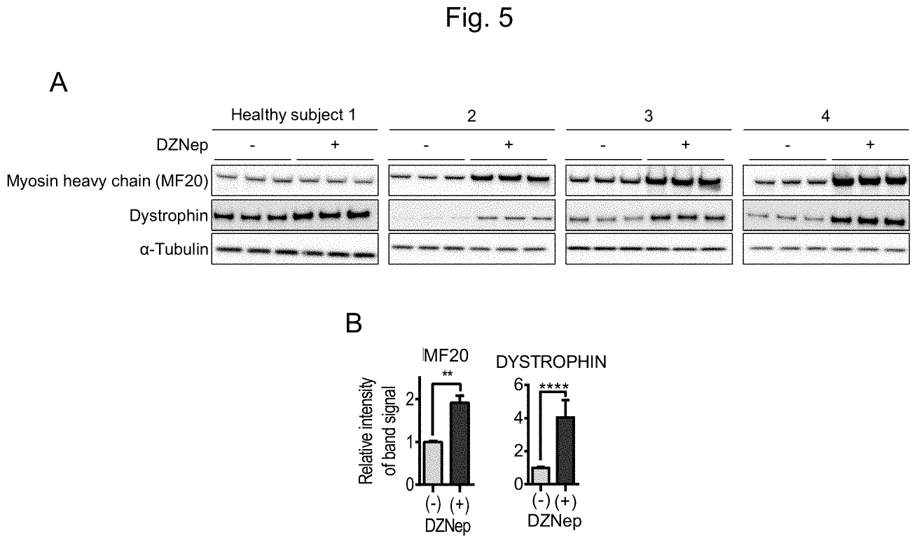

FIG. 5A shows the results of Western blotting, and FIG. 5B shows graphs showing relative intensities of band signals. As shown in FIG. 5, both the myosin heavy chain and dystrophin were found to be expressed at high levels and the myotubes were found to have been induced in the presence of DZNep by Western blotting performed with the use of the myotubes induced from urine-derived cells obtained from 4 healthy subjects. Thus, an epigenetic regulatory compound containing DZNep was found to have effects on promoting induction of the myotubes from the urine-derived cells transduced with the MYOD1. Thus, the exposing step was performed, and the preparing step was completed.

### [Example 5] In Vitro Test of an Agent Used for Exon Skipping Therapy Using the Myotubes Induced from Urine-Derived Cells Obtained from a DMD Patient

The myotubes induced from the urine-derived cells obtained from a DMD patient (i.e., the urine-cell derived myotubes) was subjected to the experiment described below in order to examine as to whether or not the therapeutic effects of an agent used for exon skipping therapy; i.e., an antisense oligonucleotide (AON), could be tested. A urine sample was obtained from a DMD patient with exon 45 deletion in the DMD gene, and the myotubes were induced from the urine-derived cells in the manner described in Examples 1 to 3. Seven days after the induction of muscular differentiation, the culture medium was replaced with a differentiation medium containing an agent used for exon skipping therapy (AON) and a 6 μM endo-porter (Gene Tools, Philomath, Oreg., U.S.A.). In addition, the medium was replaced with a medium consisting of a differentiation medium 3 days thereafter, and cells were recovered 14 days after the induction of muscular differentiation. The AON described in detail in Wilton, S. D. et al., Mol. Ther., 15, 1288-1296, 2007 was used herein. Thus, the applying step was performed.

The exon skipping efficiency was examined by RT-PCR in the manner described below. At the outset, total RNA was recovered using the RNeasy kit (Qiagen, Hilden, Germany), 1 μg of total RNA was reverse-transcribed using the cDNA reverse transcription kits (Applied Biosystems, Warrington, UK), and RT-PCR was performed using 1 μl of cDNA template, 14.9 μl of distilled water, 0.2 μl of a forward primer (10 μM), 0.2 μl of a reverse primer (10 μM), 1.6 μl of 2.5 mM dNTPs, 2 μl of 10×Ex Taq Buffer, and 0.1 μl of Ex Taq HS (Takara Bio, Shiga, Japan). The forward primer used was 5′-GCTCAGGTCGGATTGACATT-3′ (SEQ ID NO: 1), and the reverse primer used was 5′-GGGCAACTCTTCCACCAGTA-3′ (SEQ ID NO: 2). The band of the PCR product was analyzed using MultiNA (Shimadzu, Kyoto, Japan) to determine the exon skipping efficiency.

Dystrophin protein expression was analyzed by Western blotting in the same manner as described in Example 4. Also, the dystrophin protein was observed under a fluorescence microscope by immunocytochemistry as with the case of Example 4. Thus, the detecting step was performed.

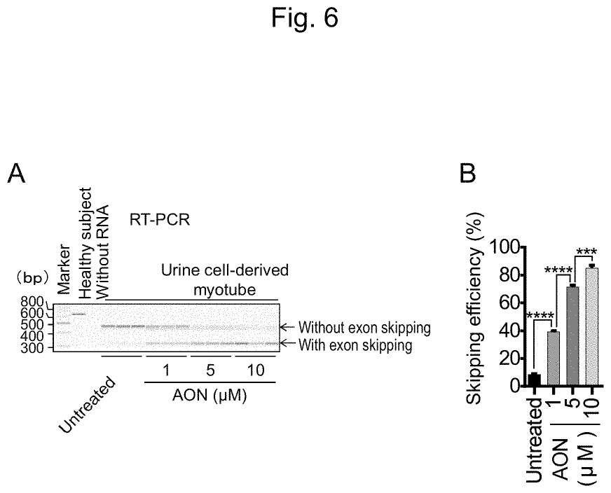

FIGS. 6 to 8 each show the results of experiments concerning the exon skipping efficiency. FIG. 6 shows dystrophin gene expression determined by RT-PCR, FIG. 6A shows a band detected by RT-PCR, and FIG. 6B shows a graph demonstrating the exon skipping efficiency determined by quantification of the band shown in FIG. 6A. In FIG. 6A, a band appearing in a sample obtained from a healthy subject indicates a full-length dystrophin gene. In the case “untreated,” a band indicating a dystrophin gene with exon 45 deletion indicated by an arrow with the term “without exon skipping” is observed. In the presence of an agent used for exon skipping therapy (AON), expression of the dystrophin gene shorter than the full-length is indicated by an arrow with the term “with exon skipping.”

The exon skipping efficiency was determined in accordance with the following equation.

Exon skipping efficiency=with exon skipping/(without exon skipping+with exon skipping)

The graph shown in FIG. 6B shows the exon skipping efficiency in terms of the mean±standard error, “***” indicates P<0.001, and “****” indicates P<0.0001.

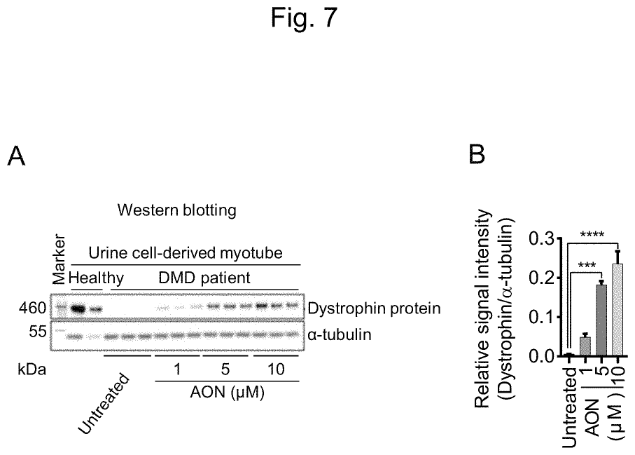

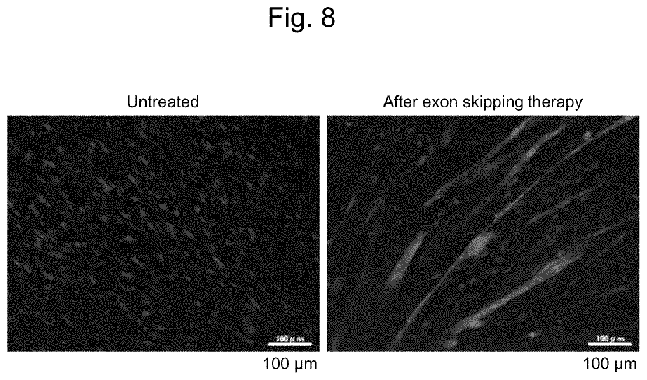

FIG. 7 and FIG. 8 show the dystrophin protein expression analyzed by Western blotting and immunocytochemistry, respectively. FIG. 7A shows the results of Western blotting, and FIG. 7B shows the graphs of the dystrophin protein levels prepared based on FIG. 7A. The graph shown in FIG. 7B shows the dystrophin protein level relative to α-tubulin in terms of the mean±standard error, “**” indicates P<0.01, “***” indicates P<0.001, and “****” indicates P<0.0001.

FIG. 8 shows the results of immunocytochemistry performed on the urine-cell derived myotubes obtained from a DMD patient and a comparison of the untreated sample and the sample after exon skipping therapy. Compared with the untreated sample, dystrophin protein (red) expression is more clearly observed in the sample after exon skipping therapy.

Thus, the detecting step was performed. As a result, it was found that AON-dose-dependent effects of exon skipping therapy could be tested at mRNA and protein levels.

### [Example 6] Establishment of a Test System that Selects a Sequence of the Optimal Agent Used for Exon Skipping Therapy for Particular DMD Gene Mutation

A urine sample was obtained from a DMD patient with exon 45-54 deletion in the DMD gene, and the urine-cell derived myotubes were induced. Seven days after the induction of muscular differentiation, the culture medium was replaced with a differentiation medium containing each antisense oligonucleotides (AON) having different sequences and a 6 μM endo-porter (Gene Tools, Philomath, Oreg., U.S.A.). In addition, the medium was replaced with a medium consisting of a differentiation medium 3 days thereafter, and, 14 days after the induction of muscular differentiation, dystrophin protein expression was semi-quantified by immunocytochemistry in the same manner as described in Example 4. The AON used was the exon-44-skipping agent, and the exon-45-skipping agent, the exon-50-skipping agent, and the exon-51-skipping agent were used for control. These AONs are described in detail in Wilton, S. D. et al., Mol. Ther., 15, 1288-1296, 2007 for the exon-44-skipping agent and the exon-45-skipping agent, Wu, B. et al., PLoS One 6, e19906, 2011 for the exon-50-skipping agent, and eteplirsen (AVI-4658) was used as the exon-51-skipping agent.

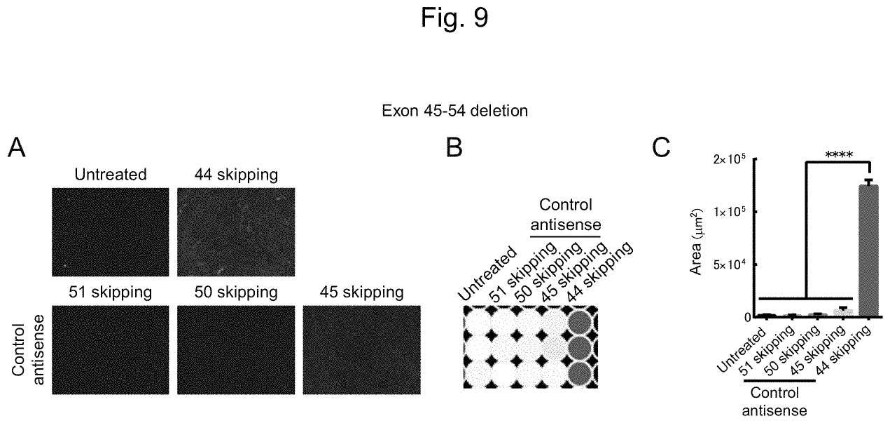

FIG. 9 shows the results of experiment. FIG. 9A shows the results of immunocytochemistry, and FIG. 9B shows a heat map for semi-quantitative analysis of fluorescence-positive regions based on FIG. 9A. FIG. 9C shows the signal intensity of the dystrophin protein determined based on FIG. 9B in terms of mean±standard error. The 1-way ANOVA test is performed (N=4 to 5), and “****” indicates P<0.0001.

FIG. 9 demonstrates that a frame-shift mutation is modified to in-frame via exon skipping and a fluorescence signal of the exon-44-skipping agent, which is deduced to express the dystrophin protein, is significantly high. Thus, it is predicted that this DMD patient would have satisfactory effects by the treatment using the exon-44-skipping agent. Thus, the identifying step is performed.

As described above, an agent used for exon skipping therapy can be tested with the use of the myotubes induced from the urine-derived cells before a particular DMD patient is subjected to actual treatment. This enables selection of a sequence of an optimal agent used for exon skipping therapy that is expected to be effective.

### SEQUENCE LISTING FREE TEXT

SEQ ID NOs: 1 and 2: artificial (synthetic oligonucleotides)

1. A method for preparing myotubes from urine-derived cells comprising:
introducing the MYOD1 gene into urine-derived cells; and
exposing the urine-derived cells to at least one of epigenetic regulatory compound.

2. The method according to claim 1, wherein, after the introducing and the exposing, the urine-derived cells comprise at least one selected from the group consisting of myoblasts and myotubes.

3. The method according to claim 1, wherein the epigenetic regulatory compound comprises at least one selected from the group consisting of a histone methyltransferase inhibitor, a histone demethylase inhibitor, a histone deacetylase inhibitor, a SIRT2 inhibitor, and a PARP inhibitor.

4. The method according to claim 3, wherein the histone methyltransferase inhibitor comprises at least one selected from the group consisting of 3-deazaneplanocin A, 3-deazaneplanocin A hydrochloride (DZNep), GSK343, SGC707, furamidine dihydrochloride, UNC2327, E7438, and MI-2 (menin-MLL inhibitor).

5. The method according to claim 3, wherein the histone demethylase inhibitor comprises at least one selected from the group consisting of IOX 1 and GSK-J1.

6. The method according to claim 3, wherein the histone deacetylase inhibitor comprises at least one selected from the group consisting of LMK-235, CAY10603, BRD73954, and VORINOSTAT.

7. The method according to claim 3, wherein the SIRT2 inhibitor comprises SirReal 2.

8. The method according to claim 3, wherein the PARP inhibitor comprises EB47.

9. The method according to claim 1, wherein, in the introducing-step, the MYOD1 gene is introduced by introduction of an expression vector comprising the MYOD1 gene under the control of an inducible promoter.

10. The method according to claim 9, wherein the expression vector further comprises a selection marker gene.

11. The method according to claim 1, wherein the urine-derived cells are derived from a patient with a muscular disease or a patient with muscular dystrophy.

12. A kit for preparing myotubes from urine-derived cells, comprising:
a means for introducing the MYOD1 gene into urine-derived cells; and
at least one of epigenetic regulatory compound.

13. The kit according to claim 12, wherein the introducing means is an expression vector used for introducing the MYOD1 gene into urine-derived cells.

14. The kit according to claim 12, wherein the epigenetic regulatory compound comprises at least one selected from the group consisting of a histone methyltransferase inhibitor, a histone demethylase inhibitor, a histone deacetylase inhibitor, a SIRT2 inhibitor, and a PARP inhibitor.

15. The kit according to claim 12, wherein the epigenetic regulatory compound comprises at least one selected from the group consisting of: 3-deazaneplanocin A and 3-deazaneplanocin A hydrochloride (DZNep); GSK343, SGC707, furamidine dihydrochloride, UNC2327, E7438 and MI-2 (menin-MLL inhibitor); IOX 1 and GSK-J1; LMK-235, CAY10603, BRD73954 and VORINOSTAT; SirReal 2, and EB47.

16. A method for testing an agent used for exon skipping therapy for a patient with muscular dystrophy comprising:
preparing myotubes from urine-derived cells obtained from a patient with muscular dystrophy by the method according to claim 1;
applying the agent used for exon skipping therapy to the myotubes; and
detecting recovery of the dystrophin mRNA and/or protein in the myotubes.

17. The method according to claim 16, wherein, in the detecting, recovery of the dystrophin mRNA and/or protein is detected by at least one method selected from the group consisting of RT-PCR, Western blotting, and immunocytochemistry.

18. The method according to claim 16, wherein the agent used for exon skipping therapy comprises at least one selected from the group consisting of an exon-44-skipping agent, an exon-45-skipping agent, an exon-50-skipping agent, an exon-51-skipping agent, and an exon-53-skipping agent.

19. A method for screening for a candidate therapeutic agent or preventive agent of a condition of inducing skeletal muscle damage comprising:
preparing myotubes from urine-derived cells obtained from a patient with a condition of inducing skeletal muscle damage by the method according to claim 1;
applying a test substance or factor to the myotubes; and
identifying the test substance or factor as the candidate therapeutic agent or preventive agent by monitoring a change in the myotubes after the applying-step.


SEQUENCE LISTING (2 sequences)

SEQ ID NO: 1 - DNA, 20 bp, Artificial
  synthetic oligonucleotide
gctcaggtcggattgacatt

SEQ ID NO: 2 - DNA, 20 bp, Artificial
  synthetic oligonucleotide
gggcaactcttccaccagta